(19) United States
(12) Patent Application Publication
(10) Pub. No.: US 2007/0055341 A1
(43) Pub. Date: Mar. 8, 2007

(54) Endograft

(75) Inventors: John K. Edoga, North Beach, NJ (US); Thierry Richard, Florham Park, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Vascular and Endovascular Surgical Technologies, Inc., Morristown, NJ (US)

(21) Appl. No.: 11/509,884

(22) Filed: Aug. 25, 2006

Related U.S. Application Data
(60) Provisional application No. 60/711,556, filed on Aug. 26, 2005.
(60) Provisional application No. 60/737,274, filed on Nov. 16, 2005.

(51) Int. Cl. A61F 2/06

(52) U.S. Cl. 623/1.11; 623/1.35

(57) ABSTRACT

An endograft deployment mechanism having a cone for placement in a blood vessel, a plurality of struts having first ends engaged with the cone and second ends opposite the first ends, and a hollow outer sheath associated with the cone, the sheath housing an endograft having proximal and distal ends, wherein the second ends of the struts are removably attached to the proximal end of the endograft such that the endograft may be separated from the struts during deployment thereof. An endograft having a trunk portion with an end, a first branch extending from the junction to an end, a second branch extending from the junction to an end, and a plurality of integral hoops disposed only within the ends, making the ends circumferentially radiopaque. The endograft may also include a stent adjacent to the junction to ensure fluid communication between the trunk and branches and opening of the bifurcation.

### CROSS-REFERENCE TO RELATED APPLICATIONS

This application claims the benefit of the filing date of U.S. Provisional Patent Application No. 60/711,556 filed Aug. 26, 2005, and 60/737,274 filed Nov. 16, 2005, the disclosures of which are hereby incorporated herein by reference.

### BACKGROUND OF THE INVENTION

There are several medical conditions which currently require surgery and/or the use of an endograft to repair body vessels, particularly arteries. For illustrative purposes, consider a patient requiring treatment in the area of the abdominal aorta above the junction of the iliac arteries due to arterial disease, such as an aneurysm.

One method of treating arterial disease in this area, for example, involves the insertion of an endograft within the existing aorta and anchoring of the graft in place such that it acts to carry blood through the afflicted portion of the aorta. This is analogous to fixing a leaking pipe by placing another pipe of smaller diameter within the existing pipe and, in essence, bypassing the afflicted area of the pipe (in this case the smaller “pipe” expands to a larger diameter matching that of the larger “pipe.”). However, the technique does have some problems, including difficulty in accurately sizing and delivering the graft in a bifurcated blood vessel. Another problem is getting both of the lower or iliac ends of the graft, which are to be disposed in the right and left iliac arteries, properly aligned and positioned while, at the same time, controlling the placement of the upper or aortic end of the graft.

While a number of techniques have been suggested, the most common techniques for unibody grafts use multiple guide wires which are inserted through the common femoral artery of one leg up into the body. A first guide wire is inserted through the common femoral artery in one leg such that its free end dangles in the aorta around the junction with the renal arteries. Another guide wire may be fed in through the same leg and crosses over from one iliac artery into the other iliac artery and out through an incision in the common femoral artery of the other leg. The loose guide wire is used to guide the entire stent and graft assembly into the abdominal aorta above the iliac divide. The aortic or proximal end of the graft is exclusively fed through the femoral artery with the two iliac ends of the graft trailing behind. Thereafter, the second guide wire, which is looped up through both iliac ends of the graft, is used in an attempt to position the crossover iliac end into proper position in the iliac artery of the other leg. Additional guide wires may also be utilized. Beside the obvious difficulties in maneuvering the device, it is difficult to ensure that the graft does not become twisted and blocked during deployment. It is also difficult to control the placement of, in particular, the iliac portion of the graft which is being maneuvered into the non-insertion iliac artery.

Similar techniques have been developed for use with modular grafts. However, such techniques also suffer from placement difficulties.

Although these devices and associated techniques have achieved some level of success in implanting grafts used to repair arterial junctions, there remains a need for further improvement.

### SUMMARY OF THE INVENTION

One aspect of the present invention provides an endograft deployment mechanism useful in endovascular surgery. The deployment mechanism preferably includes a plurality of removable struts which are housed within a deployment cone for placement into a blood vessel. The deployment cone has a proximal end and an open distal end.

The deployment mechanism further includes a hollow outer sheath trailing the deployment cone. The outer sheath preferably houses an endograft, which has a distal end and a proximal end, and which is desirably deployed within a blood vessel.

Removable struts included in the deployment mechanism to span between the deployment cone and an endograft desirably include a hook on their first end which is adapted to be removably attached to the proximal end of the endograft. The second end of the proximal struts is preferably adapted to be slidably connected to the deployment cone. The struts are preferably adapted to be collapsible within the deployment cone during insertion of the device.

A safety plug may also be included with the deployment mechanism. The safety plug is preferably adapted to slide within the outer sheath and within the endograft housed by the outer sheath. The plug is desirably adapted to engage the distal end of the deployment cone to securely cover the open end of the cone to ensure that the hooks of the struts do not cause injury to the patient as the device is removed from the body.

A further aspect of the invention provides an endograft which is useful in endovascular surgery. The endograft desirably has at least one hollow main trunk portion for placement into a blood vessel. The main portion preferably has a proximal end, a distal end, and an extended portion therebetween. The proximal end of the main trunk portion is desirably adapted to be attached to a blood vessel at its main trunk.

The endograft also preferably includes at least two hollow branches each having a proximal end, a distal end, and an extended portion therebetween. The proximal ends of the endograft branches may be connected such that they are in fluid communication with the distal end of the main trunk portion to form a junction. The distal ends of the endograft branches are desirably adapted to attach to two branches extending from the blood vessel trunk, or to endograft extensions.

Furthermore, the endograft may include a plurality of integral hoops disposed within the main trunk portion and within the plurality of endograft branches to maintain the luminal configuration of the endograft. Preferably, the hoops are included only at the three extreme ends, that of the main trunk and those of the endograft branches. These hoops are not specifically intended to promote the luminal configuration of the graft after affixation to the blood vessel or to actually affix the graft. Rather, the hoops preferably maintain the luminal openings only until the graft is affixed with staples. The endograft may also include features to make portions, particularly end portions, circumferentially radiopaque such that the features may be clearly visible through x-ray devices. Being radiopaque is preferred as unlike other endografts, the endografts as taught herein are not dependent on high residual radial force of the stents for fixation. Rather, fixation herein is dependent on virtual proximal anastomosis by staples. The staples should be placed such that the lead edge of the graft is pinned down in accordance with proper tenets of vascular anastomosis. Because stapling is the intended mode of fixation, only minimal oversizing of the endograft is needed.

While many different techniques may be employed, a further aspect of the invention provides methods for implanting a bifurcated endograft into a bifurcated blood vessel within the body of a patient. The blood vessel may be an artery with a hollow main arterial trunk, a first hollow arterial branch extending from the main arterial trunk, and a second hollow arterial branch extending from the main arterial trunk.

One method desirably includes the step of introducing an endograft deployment mechanism into the main arterial trunk to deploy a bifurcated endograft. The bifurcated endograft preferably includes a hollow main trunk portion, a first endograft branch and a second endograft branch.

The method desirably includes the steps of stapling the proximal end of the endograft to the main arterial trunk and then withdrawing the endograft deployment mechanism from the main arterial trunk. The proximal ends of both tubular endograft branch extensions are then preferably stapled to the distal end of the respective endograft branches of the endograft or to endograft extension members, in which case the distal ends of both endograft branch extensions are preferably stapled to the respective arterial branches. The stapler referenced in the application may be of the type shown and described in U.S. patent application Ser. No. 10/737,466 (published as US-2004-0176663-A1) and Ser. No. 10/837,827 (published as US-2005-0004582), the disclosures of which are hereby incorporated herein by reference

The method may also include providing an endograft deployment mechanism which includes a hollow deployment cone which houses a plurality of removable struts. The method may also include providing a hollow outer sheath housing an endograft which has a hollow main trunk portion, a first endograft branch and a second endograft branch. A safety plug may also be provided in this method.

A further aspect of the invention provides steps for preferably deploying the bifurcated endograft by displacing the deployment cone in a cephalad direction to release the removable struts at a desired level within the main arterial trunk. This method may further be performed by displacing the outer sheath in a caudad direction causing the endograft to deploy. In addition, the method may further be performed by collapsing the removable struts into the deployment cone. Finally the method may also include the step of introducing a safety plug into the main arterial trunk, which engages the open distal end of the counter-current deployment cone.

In accordance with certain aspects of the present invention an endograft deployment mechanism may comprise a deployment cone adapted to be placed in a blood vessel, the deployment cone having a proximal end and an open distal end, a plurality of struts having first ends engaged with the deployment cone and second ends opposite the first ends, and a hollow outer sheath associated with the deployment cone, the outer sheath adapted to house an endograft having a proximal end and a distal end. The second ends of the struts may be adapted to be removably attached to the proximal end of the endograft such that the endograft may be separated from the struts during deployment thereof.

The engagement of the struts with the deployment cone may be a slideable engagement.

The struts may include hooks at their second ends, the hooks being removably attached to the proximal end of the endograft.

The deployment cone and the outer sheath may include features to permit travel along a guide wire.

The deployment cone may comprise a cylindrical portion between the proximal and distal ends.

The deployment cone may taper to a point at its proximal end.

In accordance with further aspects of the present invention, an endograft suitable for being implanted into a body may comprise a trunk portion having a trunk end, a first branch extending from the trunk portion from a junction to a first branch end, a second branch extending from the trunk portion from the junction to a second branch end, and a plurality of integral hoops disposed only within the trunk end, the first branch end, and the second branch end. The hoops may be adapted to maintain a luminal configuration of the ends. A stent may be positioned adjacent to the junction, the stent adapted to ensure fluid communication through the junction and easier catheterization through the limbs.

The trunk end may include features to make the trunk end radiopaque.

The first branch end and the second branch end may include features to make the ends circumferentially radiopaque.

The first branch end and the second branch end may be adapted to be connected to extension members.

The second branch may be shorter than the first branch.

In accordance with additional aspects of the present invention, a method of implanting a bifurcated endograft into a bifurcated blood vessel, the blood vessel and the endograft having main trunks and first and second branches extending from the main trunks at common intersection points, is disclosed. The method may comprise the steps of introducing an endograft deployment mechanism into the main trunk, the deployment mechanism having a deployment cone with struts extending therefrom and an outer sheath housing the bifurcated endograft, the endograft temporarily attached to the struts at its main trunk, moving the deployment cone in a first direction relative to the outer sheath to release the struts, moving the outer sheath in a second direction relative to the deployment cone to discharge the endograft into the main trunk of the vessel, introducing a stapler into the endograft, stapling the endograft to the main trunk.

The method may further comprise permitting the branches of the endograft to enter the branches of the blood vessel, attaching the branches of the endograft to the branches of the blood vessel. The step of permitting may involve assistance.

The method may further comprise permitting the branches of the endograft to enter the branches of the blood vessel, attaching an extension graft to the first branch of the endograft, attaching the extension graft to the first branch of the blood vessel. The step of attaching the extension graft to the first branch of the blood vessel may be conducted by stapling. The step of stapling may be conducted around the circumference of the first branch of the blood vessel with at least 6 staples.

The method may further comprise spreading the struts.

The method may further comprise detaching the struts from the endograft, returning the struts into the deployment cone. Such method may further comprise introducing a safety cone through the outer sheath, inserting the safety cone into the deployment cone after returning the struts to the deployment cone.

The step of stapling may be conducted with staples having a diameter of at least 2 mm.

The step of stapling may be conducted with staples made from a memory metal.

In accordance with additional aspects of the present invention, a method of implanting a bifurcated endograft into a bifurcated blood vessel, where the blood vessel and the endograft have main trunks and first and second branches extending from the main trunks at common intersection points, and the endograft has circumferentially radiopaque reinforced ends, such method may comprise inserting the bifurcated endograft into the bifurcated blood vessel such that the trunks and branches of the bifurcated endograft and bifurcated blood vessel align, affixing the bifurcated endograft to the bifurcated blood vessel at the trunks thereof.

The method may further comprise affixing the bifurcated endograft to the bifurcated blood vessel at the branches thereof. The step of affixing may be achieved with staples.

The method may further comprise identifying the circumferentially radiopaque reinforced end of the endograft trunk with x-ray technology.

The method may further comprise affixing an extension to each branch of the endograft and affixing the extensions to the bifurcated blood vessel.

In accordance with still further aspects of the present invention, an endograft for implanting into a body may comprise a trunk portion having a trunk end, a first branch extending from the trunk portion from a junction to a first branch end, a second branch extending from the trunk portion from the junction to a second branch end. The trunk, the first branch, and the second branch may each be in fluid communication at the junction and the trunk end, the first branch end, and the second branch end may include features to make the ends radiopaque.

The radiopaque ends may be thicker than the remainder of the endograft to facilitate stapling thereof.

The endograft may further comprise three stents within the endograft, a first stent being adjacent to the trunk end, a second stent being adjacent to the first branch end, and the third stent being adjacent to the second branch end, such that the stents are away from the thicker ends.

The stents may be adapted to apply minimal or no residual pressure against an arterial wall when embedded therein.

### BRIEF DESCRIPTION OF THE DRAWINGS

A more complete appreciation of the subject matter of the present invention and the various advantages thereof can be realized by reference to the following detailed description in which reference is made to the accompanying drawings in which:

FIG. 1 is a perspective view of the endograft deployment mechanism assembly in accordance with certain aspects of the present invention;

FIG. 2 is a partial cutaway of the endograft deployment mechanism assembly in accordance with certain aspects of the present invention inserted within an abdominal aorta and at a beginning stage of the deployment process in accordance with certain aspects of the present invention;

FIG. 3 depicts a partial cutaway view of the endograft deployment mechanism assembly inserted within the abdominal aorta of FIG. 2, further depicting the deployment of the endograft through displacement of the outer sheath;

FIG. 4 depicts a partial cutaway view of the endograft deployment mechanism assembly inserted within the abdominal aorta of FIG. 3, further depicting the deployment of the endograft;

FIG. 5 depicts a partial cutaway view of the endograft deployment mechanism assembly inserted within the abdominal aorta of FIG. 4, further depicting the introduction of a stapler;

FIG. 6 depicts a partial cutaway view of the endograft deployment mechanism assembly inserted within the abdominal aorta of FIG. 5, further depicting the stapling of portions of the endograft to the abdominal aorta;

FIG. 7 depicts a partial cutaway view of the endograft deployment mechanism assembly inserted within the abdominal aorta of FIG. 6, further depicting the stapling of the endograft to the abdominal aorta;

FIG. 8 depicts a partial cutaway view of the endograft deployment mechanism assembly inserted within the abdominal aorta of FIG. 7, further illustrating the collapsing of the proximal struts into the deployment cone;

FIG. 9 depicts a partial cutaway view of the endograft deployment mechanism assembly inserted within the abdominal aorta of FIG. 8, further illustrating the introduction of the safety plug into the outer sheath;

FIG. 10 depicts a partial cutaway view of the endograft deployment mechanism assembly inserted within the abdominal aorta of FIG. 9, further depicting the placement of the safety plug in the abdominal aorta;

FIG. 11 depicts a partial cutaway view of the endograft deployment mechanism assembly inserted within the abdominal aorta of FIG. 10, showing the safety plug engaging the open distal end of the deployment cone;

FIG. 12 depicts a partial cutaway view of the endograft deployment mechanism assembly inserted within the abdominal aorta of FIG. 11, further illustrating the introduction of a first branch extension into the ipsilateral limb of the abdominal aorta;

FIG. 13 depicts a partial cutaway view of the endograft deployment mechanism assembly inserted within the abdominal aorta of FIG. 12, further illustrating the stapling of the first branch extension to a branch of the endograph;

FIG. 14 depicts a partial cutaway view of the endograft deployment mechanism assembly inserted within the abdominal aorta of FIG. 13, further illustrating the introduction of a second branch extension into the contralateral limb of the abdominal aorta;

FIG. 15 depicts a partial cutaway view of the endograft deployment mechanism assembly inserted within the abdominal aorta of FIG. 14, further illustrating the stapling of the second branch extension to a branch of the endograph;

FIG. 16 depicts a partially cutaway perspective view of the completed endograft replacement in accordance with certain aspects of the present invention;

FIG. 17 is a partially cutaway perspective view of the abdominal area which illustrates the initial steps of deployment of an endograft with an endograft deployment mechanism featuring an in-stream nose cone in accordance with further aspects of the present invention;

FIG. 18 depicts a partially cutaway perspective view of the endograft deployment mechanism assembly inserted within the abdominal aorta of FIG. 17, further illustrating the use of an in-stream nose cone;

FIG. 19 is a perspective view of an endograph illustrating the use of a reinforced circumferentially radiopaque region of the endograft in accordance with another additional aspect of the present invention; and,

FIG. 20 is a perspective view of an endograft body in accordance with further aspects of the present invention.

### DETAILED DESCRIPTION

In the following is described certain embodiments of the endograft deployment mechanism of the present invention. In describing the embodiments illustrated in the drawings, specific terminology will be used for the sake of clarity. However, the invention is not intended to be limited to the specific terms so selected, and it is to be understood that each specific term includes all technical equivalents that operate in a similar manner to accomplish a similar purpose.

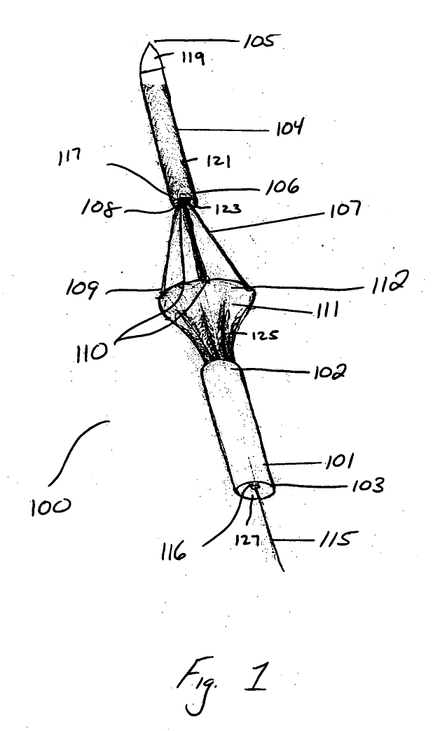

In accordance with one embodiment of the present invention, an endograft deployment mechanism is used to carry a modular bifurcated graft to a desired location within a bifurcated blood vessel, such as the abdominal aorta. FIG. 1, which along with the other figures is not to scale, illustrates one embodiment of an endograft deployment mechanism assembly 100 in accordance with certain aspects of the present invention.

In the embodiment of the present invention depicted in FIG. 1, assembly 100 is intended for deployment of an endograft into a patient utilizing a guide wire 115. The elements of assembly 100 are generally described as being “proximal” or “distal” depending on their relative position with respect to the head and feet of the patient. When a member is referred to as having a “proximal”, or “upper” portion and a “distal” or “lower” portion, the “proximal” or “upper” portion shall generally refer to the portion closest to patient's head and the “distal” or “lower” portion shall generally refer to the portion closest to the patient's feet. Referring to elements as being “proximal”, “distal”, etc. is for ease of reference purposes only, and should not be construed as requiring a specific location or direction with respect to the position of the patient's body. Similarly, the displacement of the elements in assembly 100 is generally described as being in a “cephalad” or “caudad” direction. When an element is displaced in a “cephalad” direction, it is displaced toward the head. When an element is displaced in a “caudad” direction, it is displaced toward the posterior end of the body or away from the head. Of course, other arrangements are possible.

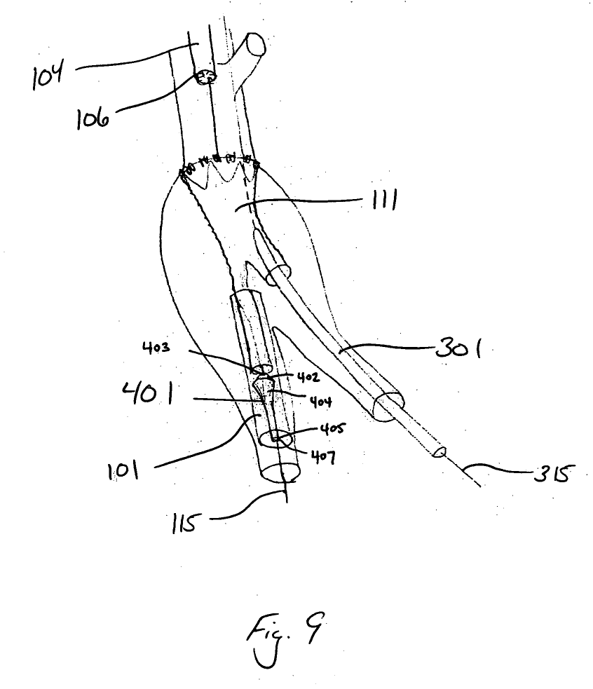

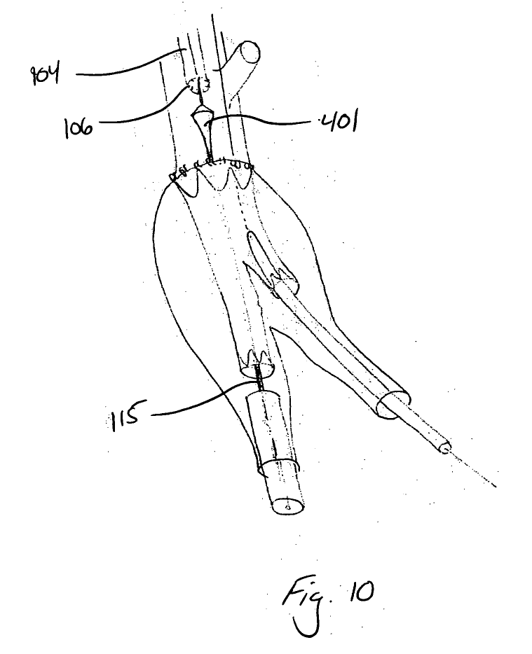

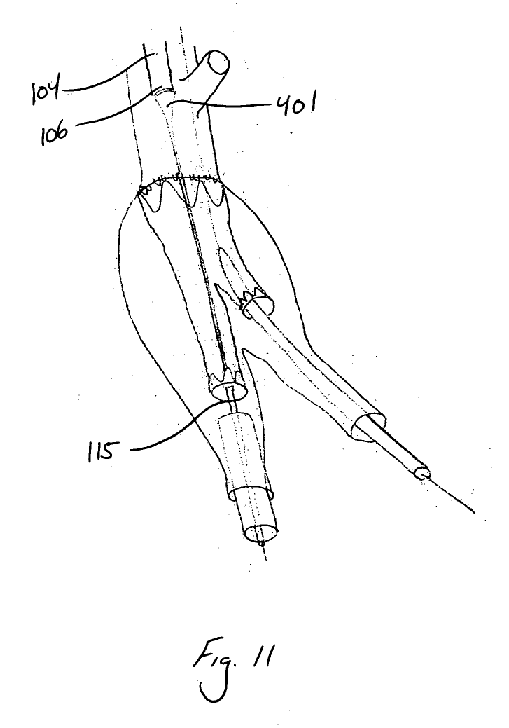

In accordance with one particular aspect of the invention, assembly 100 includes an outer sheath 101, having a proximal end 102 and a distal end 103. Outer sheath 101 is preferably constructed in a cylindrical manner with a hollow internal passage 127 adapted to house an endograft 111. In other embodiments, outer sheath 101 may be constructed in an octagonal, square, or other manner suitable for insertion into a blood vessel. The ends of outer sheath 101 are open to allow for deployment of endograft 111 and to allow for passage of a safety plug 401 (shown more clearly in FIGS. 9-11 and more fully described in the accompanying text). Outer sheath 101 also includes an internal cylinder 116 running along its longitudinal axis, which is adapted to accept the guide wire 115 in the conventional manner. Outer sheath 101 may be constructed of biocompatible materials such as plastic, stainless steel, or titanium, or other materials which are compatible with insertion within the human body.

Assembly 100 also includes a deployment cone 104, which has a proximal end 105 and a distal end 106. Deployment cone 104 is comprised of a first section 119, nearest proximal end 105 and a second section 121 nearest distal end 106, the second section being cylindrical with a hollow internal passage 123 adapted to house removable struts 107. The first section 119 tapers from a cylindrical construction to a pointed end in the geometry of a cone at its extreme proximal end 105. In other embodiments, deployment cone 104 may also be constructed in an octagonal or other manner suitable for insertion into a blood vessel. The proximal end 105 of deployment cone 104 may be completely closed or, preferably, may have an opening adapted to allow the guide wire 115 to pass through. The distal end 106 of deployment cone 104 is open to allow for the housing and deployment of the removable struts 107 and to accept the guide wire 115. Deployment cone 104 preferably includes a mechanism for slidable connection with the removable struts 107 within its internal hollow passage 123. An example of such mechanism includes, but is not limited to, the use of longitudinal grooves located on the inner surface of the internal hollow passage 123 which secure the struts and allow longitudinal travel but prevent the struts 107 from being completely detached from the deployment cone 104. In this regard, the struts 107 may include end features adapted to fit and slide within the longitudinal grooves, but which are held in the groove by the shoulders thereof.

Deployment cone 104 may also include an internal cylinder 117 running along its longitudinal axis, the cylinder being adapted to accept guide wire 115. Deployment cone 104 may be constructed of material such as plastic, stainless steel, titanium, or other material which is compatible with insertion within the human body.

Although not shown, when assembly 100 is in a collapsed position, proximal end 102 of outer sheath 101 engages the distal end 106 of the counter-current deployment cone 104. These elements may be removably connected to one another or may abut one another without being connected.

Assembly 100 also includes a plurality of removable struts 107 which may be temporarily housed within the hollow deployment cone 104. Each strut 107 has a proximal end 108 and a distal end 109. Removable struts 107 are preferably rigid and may be constructed of material such as plastic, stainless steel, or titanium. The material must also be compatible with insertion within the human body. In other embodiments, the removable struts 107 may be flexible in nature and/or constructed of memory metals. In the preferred embodiment, the removable struts 107 include hooks 110 on their distal ends 109. Hooks 110 are adapted to be removably attached to the proximal end 112 of the endograft 111. In other embodiments, the removable struts 107 may be removably attached to endograft 111 through the use of bonding material or other features to permit their attachment to each other. In the preferred embodiment, removable struts 107 slidably connect to the deployment cone 104 within its internal hollow passage 123, as previously discussed.

The endograft 111 included in assembly 100 may be used to repair a blood vessel, such as the abdominal aorta above the junction of the iliac arteries. In such case, endograft 111 may be bifurcated to include two branches extending off a main trunk. In other embodiments, the endograft 111 may also contain more then two branches.

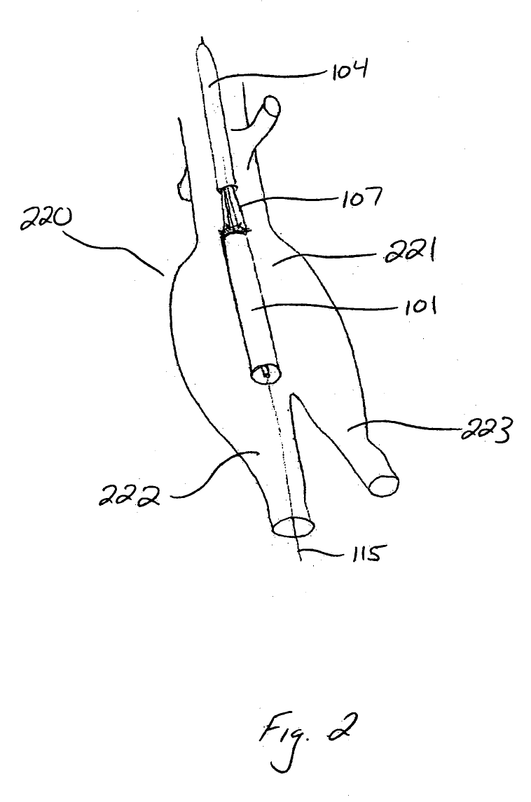

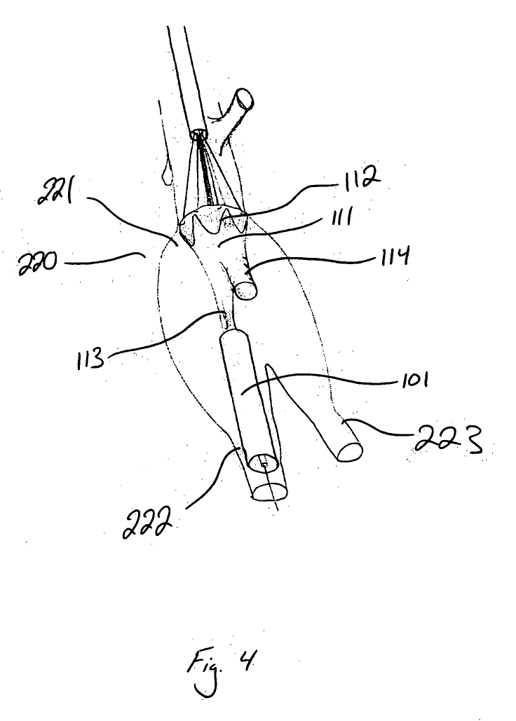

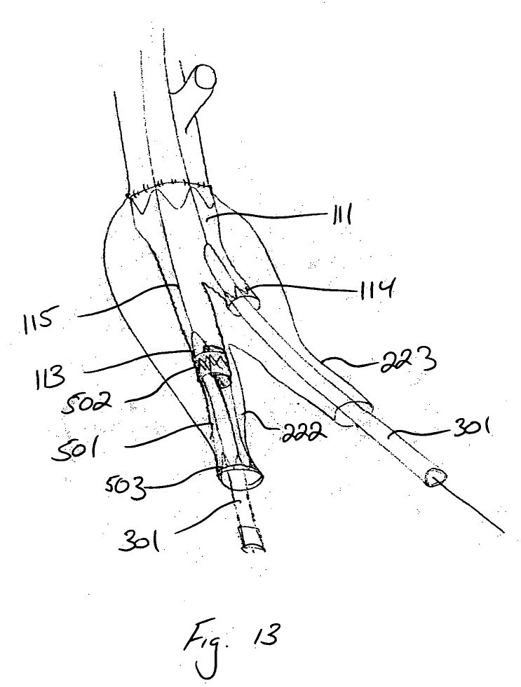

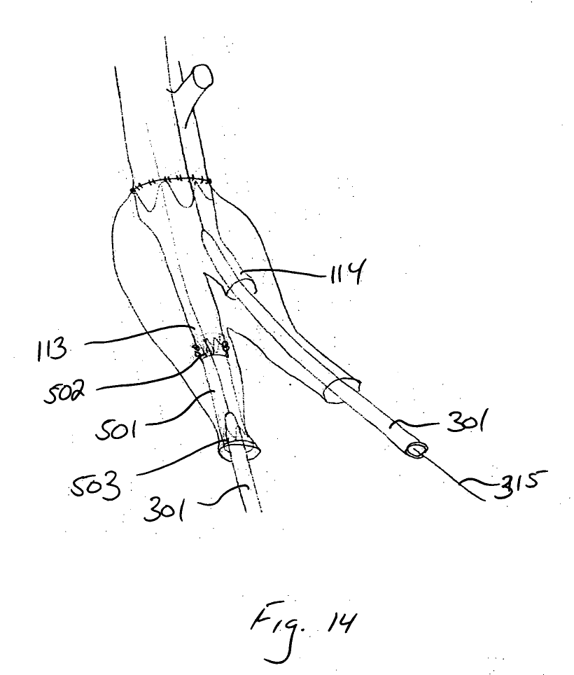

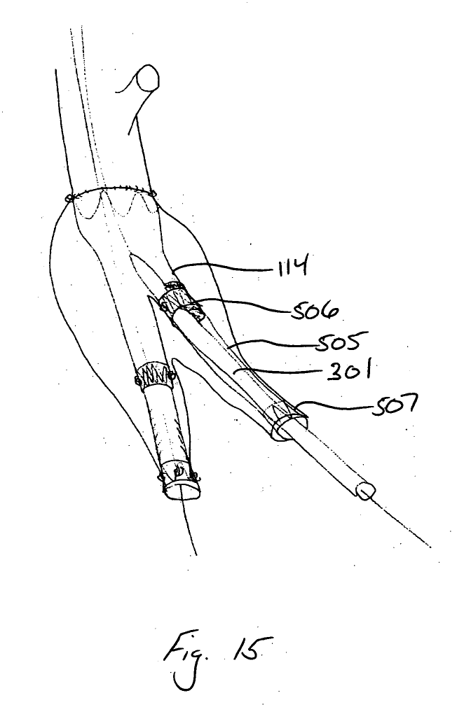

In the preferred embodiment, endograft 111 desirably has at least one hollow main trunk portion 125 for placement into a blood vessel. The main trunk portion 125 preferably has a proximal end 112 and, a distal end (not shown in FIG. 1). The proximal end 112 of the main trunk portion 125 of endograft 111 is desirably adapted to be attached to a blood vessel, such as the abdominal aorta shown in FIG. 2, at its main trunk 221. The endograft also preferably includes at least two hollow branches 113, 114 (shown in more detail in FIGS. 4-16), each having a proximal end, a distal end, and an extended portion therebetween. The proximal ends of the branches may be connected such that they are in fluid communication with the distal end of the main trunk portion 125 to form a junction. The distal ends of the branches may be adapted to attach to the two arterial branches 222, 223 extending from the main arterial trunk 221, namely the ipsilateral limb and the contralateral limb of the abdominal aorta. In preferred embodiments, however, the distal ends of the branches are adapted to be secured to extension members, as will be discussed. Furthermore, endograft 111 may include a plurality of integral hoops (i.e. stents) disposed within the main trunk portion 125 and within the plurality of endograft branches 113, 114 to maintain the luminal configuration of endograft 111. Such hoops may be manufactured as stainless steel stents, as known generally in the art, or in other configurations.

Preferably, the hoops are included only at the extreme ends of the endograft 111, rather than throughout the entire structure. In such an arrangement, the endograft 111 will include only three (3) such hoops, provided the endograft comprises a main trunk and two branches. When arranged as such, the endograft remains quite flexible throughout its main structure, yet retains the full luminal diameter at its ends.

Endograft 111 is temporarily housed within the internal passage 127 of outer sheath 101 but is preferably not connected to outer sheath 101 to facilitate deployment within the blood vessel. The endograft 111 must be constructed of biocompatible material. As well known in the industry, such grafts may be formed from expanded polytetrafluroethylene (PTFE), teflon, or polyester, among other materials.

FIG. 2 depicts a partially cutaway view of an endograft deployment mechanism assembly in accordance with certain aspects of the present invention in an initial stage in a method of introducing a bifurcated endograft into a patient, where assembly 100 is inserted into the general area of an arterial junction, such as the abdominal aorta 220, via a guide wire 115 to deploy endograft ill which is housed within the internal passage 127 of outer sheath 101. Abdominal aortas 220 include a hollow main arterial trunk 221, a first hollow arterial branch (ipsilateral limb) 222 extending from the main arterial trunk 221, and a second hollow arterial branch (centralateral limb) 223 extending from the main arterial trunk 221 such that all branches are in fluid communication with each other.

In order to implant the endograft 111, assembly 100 is inserted in the abdominal aorta 220 through a first access point and then along guide wire 115 to the interior of main arterial trunk 221, via first arterial branch 222. Deployment cone 104 is then displaced in a cephalad direction releasing removable struts 107 at a desired level within main arterial trunk 221. This configuration is shown in FIG. 2.

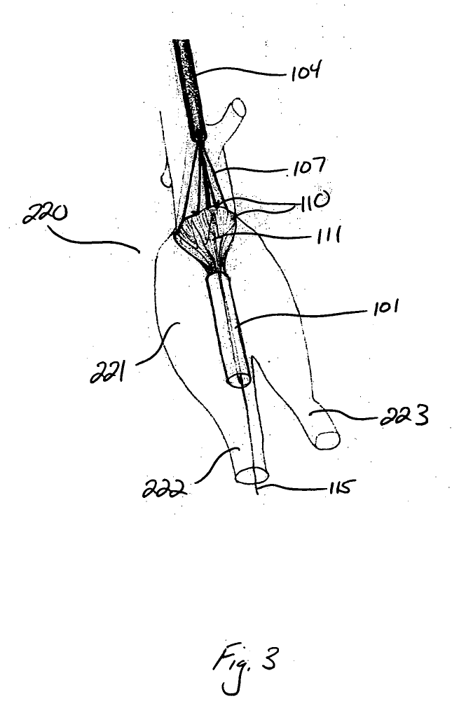

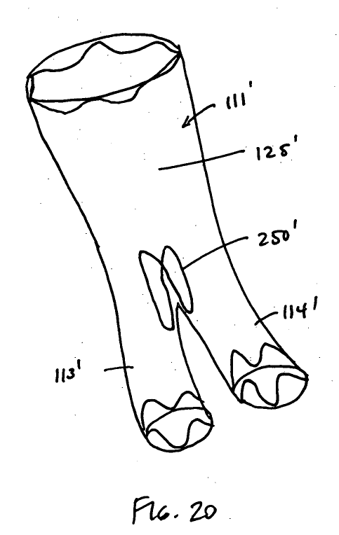

FIG. 3 and FIG. 4 depict outer sheath 101 being displaced in a caudad direction. Caudad displacement of the outer sheath 101 causes the endograft 111, which is attached to hooks 110 of removable struts 107, to deploy and exposes the main trunk 125 and branches 113, 114 of endograft 111. The open proximal end of endograft 111 may maintain its luminal configuration through the use of integral hoops within endograft 111 and/or by the fanning out of removable struts 107 once the struts and endograft clear their respective housings. In addition, as shown in FIG. 20, an endograft 111′ may be utilized which includes an integral stent 250′ adjacent to the junction of the main trunk portion 125′ and branches 113′, 114′. The stent 250′ may be V-shaped, in a similar fashion as butterfly wings, and not tubular. The purpose of the stent 250′ is to maintain fluid communication between the main trunk portion 125′ and the branches 113′, 114′. This is achieved by ensuring that the walls of the endograft 111′ in the vicinity of the junction does not collapse once the endograft 111′ is deployed and to ensure easier catheterization through the limbs.

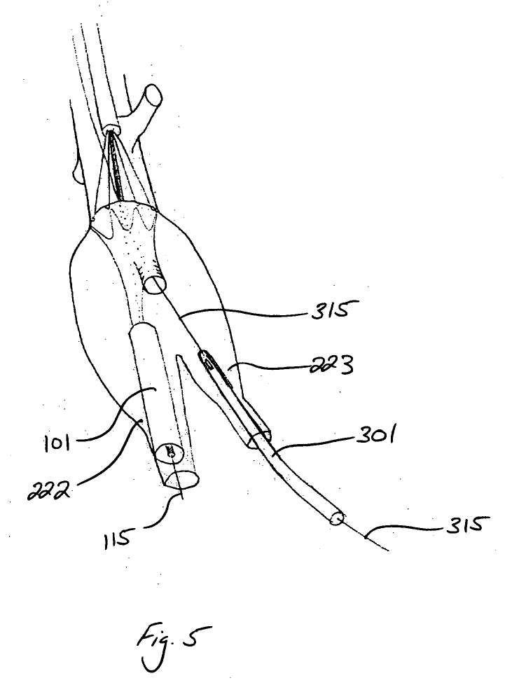

A non occlusive stapler 301 is inserted via a second guide wire 315, as depicted in FIGS. 5-7, and is used to staple the proximal end 112 of the main trunk portion 125 of endograft 111 to main arterial trunk 221 such that these elements are in fluid communication with each other without leakage. The stapler referenced in the application may be of the type shown and described in U.S. patent application Ser. Nos. 10/737,466 and 10/837,827, the disclosures of which are hereby incorporated herein by reference. Other staplers may also be utilized. The staples utilized are preferably chosen from among those disclosed in U.S. patent application Ser. No. 10/837,827, and are preferably approximately 7 mm in diameter for connection with the main arterial trunk 221, or 2 mm or less in diameter for connection with a branch 222, 233. Notwithstanding, staples measuring 2 mm, 3 mm, 4 mm, 5 mm, 6 mm, 7 mm, 8 mm, 9 mm, 10 mm or even greater diameters or fractions thereof may be utilized. Preferably, the staples are sized to properly maintain anastomosis. Further, approximately 8-10 staples are preferred at each location. Notwithstanding, less than 8 staples, for example 1, 2, 3, 4, 5, 6, or 7 staples may be utilized. Additionally, greater than 10 staples may be utilized, if desired.

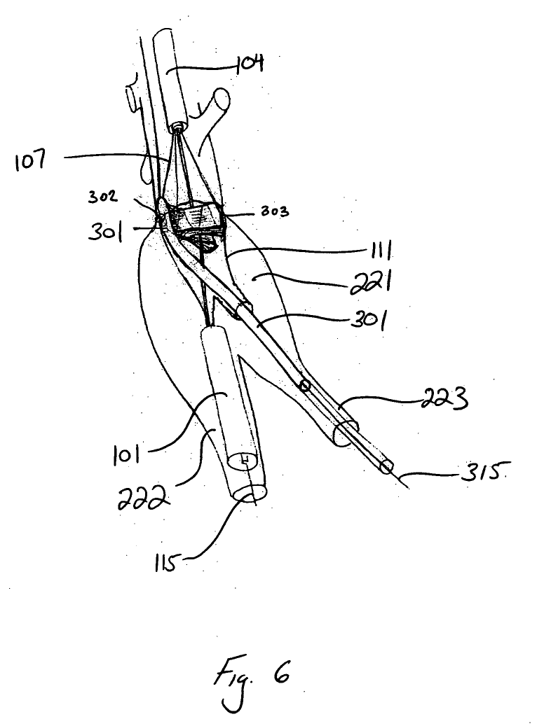

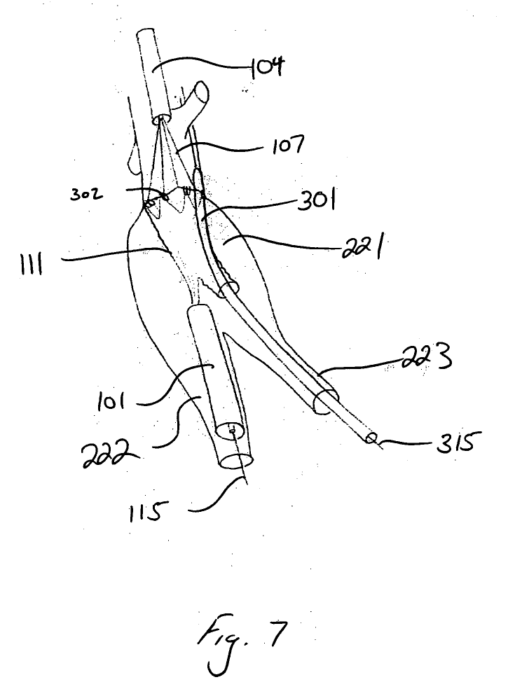

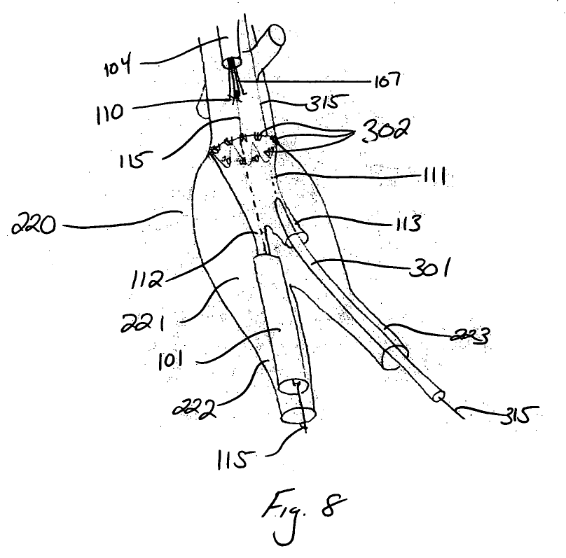

As shown in FIG. 5, and as previously discussed, the stapler 301 may be inserted into the second branch 223 along a second guide wire 315. In FIG. 6, it is shown that the stapler 301 may include a nonoccluding biasing mechanism, such as a bifurcated balloon 303, which may be utilized to ensure that the stapler is properly positioned for firing. Once staple 302 is discharged, the biasing mechanism may be retracted, the stapler 301 rotated to the next position, and the biasing mechanism redeployed, as taught in U.S. patent application Ser. Nos. 10/737,466 and 10/837,827. A subsequent staple 302 may then be deployed, as shown in FIG. 7. FIG. 8 depicts a series of staples 302 deployed to connect the endograft 111 to the abdominal aorta 220. In other embodiments, a stapler capable of firing multiple staples simultaneously may also be utilized. For example, a stapler such as that disclosed in Application No. 60/737,274 may be utilized to fire a number of staples while deploying the endograft simultaneously. A second stapler may then follow to complete the anastomosis, as necessary.

FIG. 8 also depicts removable struts 107 being collapsed into deployment cone 104 after the staples 302 have been used to attach main trunk portion 112 of the endograft 111 to main arterial trunk 221 by stapler 301.

FIGS. 9-11 depict further steps involved with certain alternate aspects of the present invention, which include introduction of a safety plug 401 into the main arterial trunk 221 over a guide wire 115. Safety plug 401 is comprised of a first section 402 at its proximal end 403 and a second section 404 at its distal end 405. The second section 404 is preferably conical in shape tapering from a larger diameter at its limit nearest the first section 402 to a smaller diameter at its extreme distal end 405. The first section 402 is preferably shorter in length then the second section 404, and is also preferably conical in shape tapering from a wider diameter at its limit nearest the second section 404, which is the same diameter as that portion of the second section, to a smaller diameter at its extreme proximal end 403. In addition, safety plug 401 may include a cylindrical passage 407 along its longitudinal axis adapted to accept guide wire 115. The safety plug 401 may be advanced along guide wire 115 to engage the open distal end 106 of the deployment cone 104 to prevent hooks 110 from injuring the patient. The deployment cone 104 may then be removed from the patient.

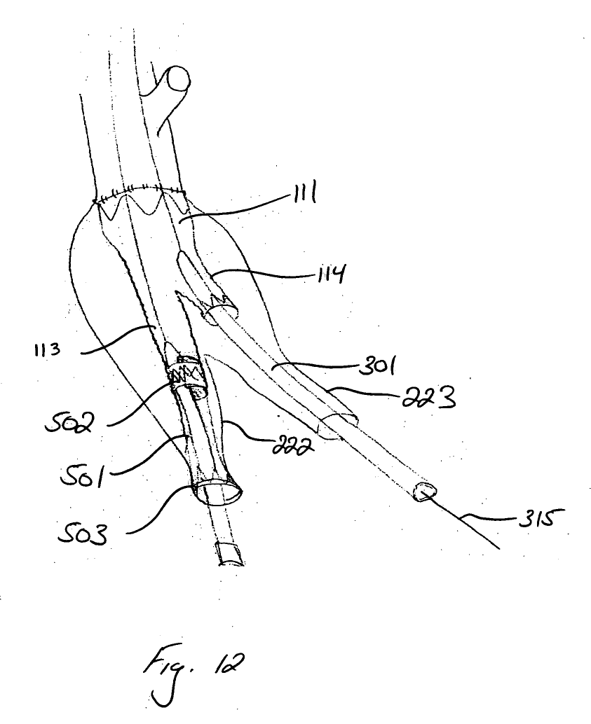

After the deployment cone 104 is removed, tubular branch extensions 501, 505 may be introduced as depicted in FIGS. 12-15. Tubular branch extensions 501, 505 include a hollow internal passage and are constructed in a cylindrical manner to match that of branches 113, 114. The proximal end 502 of the first branch extension 501 is stapled or otherwise connected onto the distal end of the first branch 113 of the endograft 111 such that these elements are in fluid communication with each other without leakage. The distal end 503 of the first branch extension 501 is thereafter stapled to the first arterial branch 222 such that these elements are in fluid communication with each other without leakage. Stapling may be conducted in a similar manner as described above. In addition, it will be appreciated that in preferred embodiments, the proximal end 502 and distal end 503 of the branch extension 501 may include integral hoops to retain the full luminal diameter of the branch extension at its ends.

The proximal end 506 of the second tubular branch extension 505 is then stapled onto the distal end of the second branch 114 such that these elements are in fluid communication with each other without leakage. The distal end 507 of the second branch extension 505 is thereafter stapled to the second arterial branch 223 such that these elements are in fluid communication with each other without leakage. Again, stapling may be conducted in a similar manner as described above. Also, the second branch extension 505 may include hoops at its ends 506, 507, which are similar to those of the first branch extension 501.

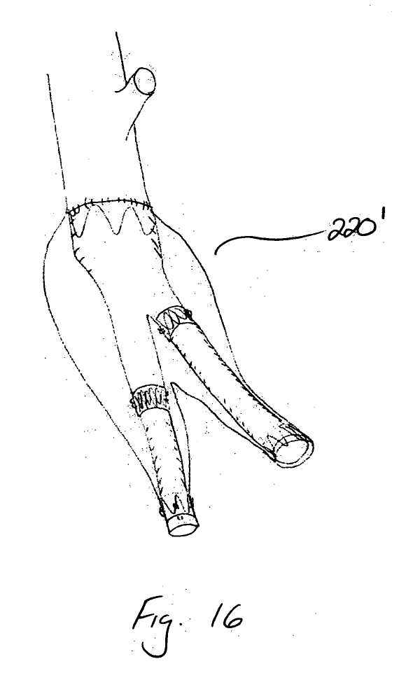

FIG. 16 depicts a completed endograft aortic replacement 220′, where blood may flow through the endograft to avoid damaged or otherwise compromised areas of the abdominal aorta.

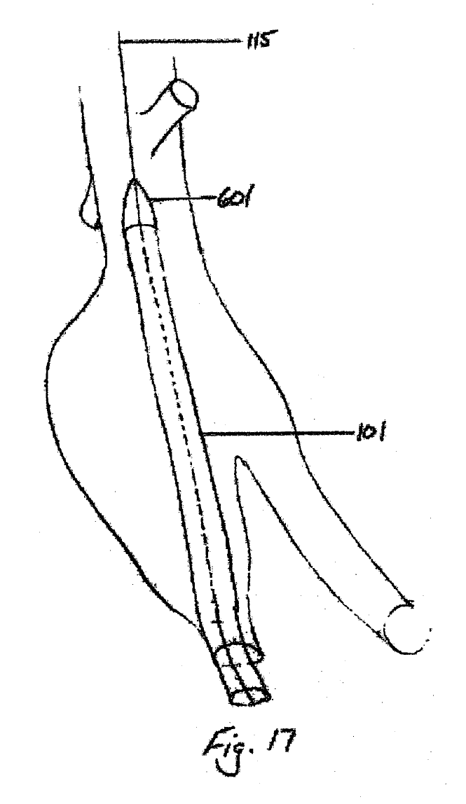

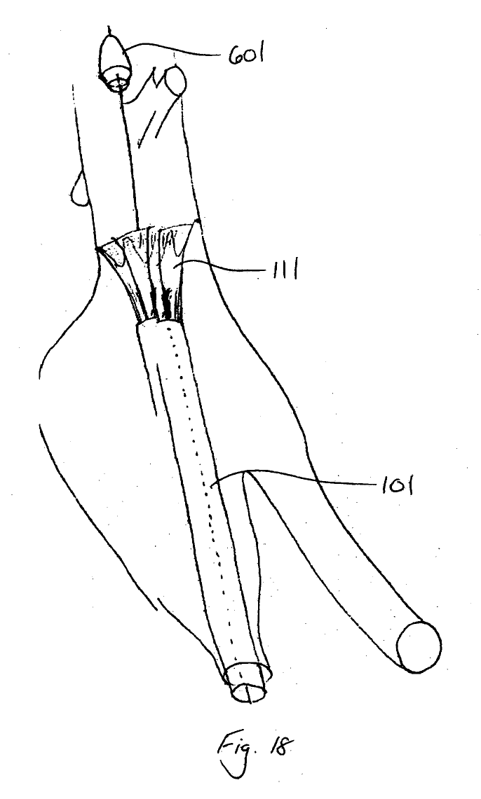

FIG. 17 depicts an initial step in another method of implanting an endograft in accordance with the present invention. In this method, a nose cone 601 containing an endograft 111 may be deployed into the abdominal aorta 220 along guide wire 115, together with an attached outer sheath 101. The nose cone 601 and outer sheath 101 may then be separated, as shown in FIG. 18, with the nose cone being moved in the cephalad direction and/or the outer sheath being moved in the caudad direction. Once separated, the endograft 111, by virtue of the integral hoops (preferably located only at the ends) and flow of blood through to abdominal aorta, will expand as shown in FIG. 18. Once in this position, the outer sheath 101 may be removed completely and the endograft 111 stapled to the abdominal aorta in the manner previously described.

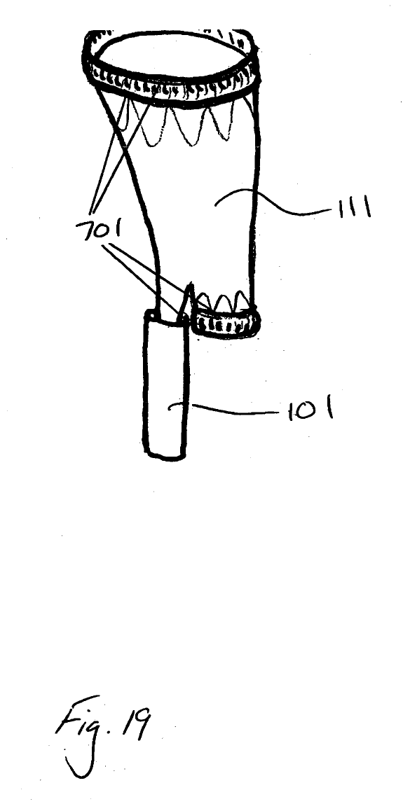

FIG. 19 depicts reinforced circumferentially radiopaque ends 701 of endograft 111, in accordance with other aspects of the present invention. Reinforcement of the endograft 111 may be accomplished in multiple ways, including, but not limited to, doubling the endograft layers and through the use of a pre-formed biocompatible web attached to the ends of each opening of endograft 111. The preferred reinforcement also includes features suitable to make the reinforcement circumferentially radiopaque, such that the limits may be identified by X-ray. Suitable radiopaque materials are well-known in the industry. Furthermore, the area of reinforcement is preferably free of metal struts of a stent.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An endograft deployment mechanism, said mechanism comprising:
a deployment cone adapted to be placed in a blood vessel, said deployment cone having a proximal end and an open distal end;
a plurality of struts having first ends engaged with said deployment cone and second ends opposite said first ends; and,
a hollow outer sheath associated with said deployment cone, said outer sheath adapted to house an endograft having a proximal end and a distal end;
wherein, said second ends of said struts are adapted to be removably attached to the proximal end of the endograft such that the endograft may be separated from the struts during deployment thereof.

2. The endograft deployment mechanism of claim 1, wherein said engagement of said struts with said deployment cone is a slideable engagement.

3. The endograft deployment mechanism of claim 1, wherein said struts include hooks at their second ends, said hooks being removably attached to said proximal end of said endograft.

4. The endograft deployment mechanism of claim 1, wherein said deployment cone and said outer sheath include features to permit travel along a guide wire.

5. The endograft deployment mechanism of claim 1, wherein said deployment cone comprises a cylindrical portion between said proximal and distal ends.

6. The endograft deployment mechanism of claim 1, wherein said deployment cone tapers to a point at its proximal end.

7. An endograft for implanting into a body, said endograft comprising:
a trunk portion having a trunk end;
a first branch extending from said trunk portion from a junction to a first branch end;
a second branch extending from said trunk portion from said junction to a second branch end; and,
a plurality of integral hoops disposed only within said trunk end, said first branch end, and said second branch end, said hoops adapted to maintain a luminal configuration of said ends;
a stent positioned adjacent to said junction, said stent adapted to ensure fluid communication through said junction and opening of the bifurcation.

8. The endograft of claim 7, wherein said trunk end includes features to make said trunk end circumferentially radiopaque.

9. The endograft of claim 7, wherein said first branch end and said second branch end include features to make said ends circumferentially radiopaque.

10. The endograft of claim 7, wherein said first branch end and said second branch end are adapted to be connected to extension members.

11. The endograft of claim 7, wherein said second branch is shorter than said first branch.

12. A method of implanting a bifurcated endograft into a bifurcated blood vessel, said blood vessel and said endograft having main trunks and first and second branches extending from the main trunks at common intersection points, said method comprising the steps of:
introducing an endograft deployment mechanism into said main trunk, the deployment mechanism having a deployment cone with struts extending therefrom and an outer sheath housing the bifurcated endograft, the endograft temporarily attached to the struts at its main trunk;
moving the deployment cone in a first direction relative to the outer sheath to release the struts;
moving the outer sheath in a second direction relative to the deployment cone to discharge the endograft into the main trunk of the vessel;
introducing a stapler into the endograft;
stapling the endograft to the main trunk.

13. The method claim 12, further comprising:
permitting the branches of the endograft to enter the branches of the blood vessel;
attaching the branches of the endograft to the branches of the blood vessel.

14. The method of claim 13, wherein said step of permitting involves assistance.

15. The method of claim 12, further comprising:
permitting the branches of the endograft to enter the branches of the blood vessel;
attaching an extension graft to the first branch of the endograft;
attaching the extension graft to the first branch of the blood vessel.

16. The method of claim 15, wherein said step of attaching the extension graft to the first branch of the blood vessel is conducted by stapling.

17. The method of claim 16, wherein said step stapling is conducted around the circumference of the first branch of the blood vessel with at least 6 staples.

18. The method of claim 12, further comprising:
spreading the struts.

19. The method of claim 12, further comprising:
detaching the struts from the endograft;
returning the struts into the deployment cone.

20. The method of claim 19, further comprising:
introducing a safety cone through the outer sheath;
inserting the safety cone into the deployment cone after returning the struts to the deployment cone.

21. The method of claim 12, wherein said step of stapling is conducted with staples having a diameter of at least 2 mm.

22. The method of claim 12, wherein said step of stapling is conducted with staples made from a memory metal.

23. A method of implanting a bifurcated endograft into a bifurcated blood vessel, said blood vessel and said endograft having main trunks and first and second branches extending from the main trunks at common intersection points, the endograft having circumferentially radiopaque reinforced ends, said method comprising the steps of:
inserting the bifurcated endograft into the bifurcated blood vessel such that the trunks and branches of the bifurcated endograft and bifurcated blood vessel align;
affixing the bifurcated endograft to the bifurcated blood vessel at the trunks thereof.

24. The method of claim 23, further comprising affixing the bifurcated endograft to the bifurcated blood vessel at the branches thereof.

25. The method of claim 23, wherein said step of affixing is achieved with staples.

26. The method of claim 23, further comprising identifying the circumferentially radiopaque reinforced end of the endograft trunk with x-ray technology.

27. The method of claim 23, further comprising affixing an extension to each branch of the endograft and affixing the extensions to the bifurcated blood vessel.

28. An endograft for implanting into a body, said endograft comprising:
a trunk portion having a trunk end;
a first branch extending from said trunk portion from a junction to a first branch end;
a second branch extending from said trunk portion from said junction to a second branch end; and,
wherein said trunk, said first branch, and said second branch are each in fluid communication at said junction and said trunk end, said first branch end, and said second branch end include features to make said ends radiopaque.

29. The endograft of claim 28, wherein said radiopaque ends are thicker than the remainder of said endograft to facilitate stapling thereof.

30. The endograft of claim 29, further comprising three stents within said endograft, a first stent being adjacent to said trunk end, a second stent being adjacent to said first branch end, and the third stent being adjacent to said second branch end, such that said stents are away from the thicker ends.

31. The endograft of claim 30, wherein said stents are adapted to apply minimal or no residual pressure against an arterial wall when embedded therein.
